SEQUENCE LISTING (9 sequences)

SEQ ID NO: 1 - protein, 24 aa, Conus radiatus
  PEPTIDE (1)..(24): Xaa is Pro or hydroxy-Pro (O)
LXSCCSLNLRLCXVXACKRNXCCT

SEQ ID NO: 2 - DNA, 87 bp, Conus radiatus
  CDS (1)..(84)
gaaaagagactaccatcgtgttgctcccttaacttgcggctttgcccagtaccagcatgcaaacgtaacccttgttgcacaggataa

SEQ ID NO: 3 - protein, 28 aa, Conus radiatus
EKRLPSCCSLNLRLCPVPACKRNPCCTG

SEQ ID NO: 4 - protein, 39 aa, Oncorhynchus mykiss
EAGSENSFFKSIPDAFWWAVVTMTTVGYGDMTPVGVWGK

SEQ ID NO: 5 - protein, 39 aa, Oncorhynchus mykiss
EADEPESQFESIPDAFWWAVVSMTTVGYGDMVPTTIGGK

SEQ ID NO: 6 - protein, 22 aa, Conus geographus
  PEPTIDE (1)..(22): Xaa is hydroxy-Pro (O)
RDCCTXXKKCKDRQCKXQRCCA

SEQ ID NO: 7 - protein, 21 aa, Conus purpurascens
  PEPTIDE (1)..(21): Xaa is hydroxy-Pro (O)
ZRCCGFXKSCRSRQCKXHRCC

SEQ ID NO: 8 - protein, 23 aa, Conus purpurascens
  PEPTIDE (1)..(23): Xaa is hydrosy-Pro (O)
HXCCLYGKCRRYXGCSSASCCQR

SEQ ID NO: 9 - protein, 27 aa, Conus purpurascens
  PEPTIDE (1)..(27): Xaa is hydroxy-Pro (O)
CRIXNQKCFQHLDDCCSRKCNRFNKCV

(19) United States
(12) Patent Application Publication
(10) Pub. No.: US 2008/0274976 A1
(43) Pub. Date: Nov. 6, 2008

(54) USES OF NOVEL POTASSIUM CHANNEL BLOCKERS

(75) Inventors: Baldomero OLIVERA, Salt Lake City, UT (US); Maren WATKINS, Salt Lake City, UT (US); Heinrich TERLAU, Eckernfoerde (DE); Michael FERBER, Adelebsen (DE)

Correspondence Address: ROTHWELL, FIGG, ERNST & MANBECK, P.C., 1425 K STREET, N.W., SUITE 800, WASHINGTON, DC, 20005, US

(73) Assignees: UNIVERSITY OF UTAH RESEARCH FOUNDATION, Salt Lake City, UT (US); MAX-PLANCK-GESELLSCHAFT zur FORDERUNG der WISSENSCHAFTEN e.V., Muenchen (DE)

(21) Appl. No.: 12/029,231

(22) Filed: Feb. 11, 2008

Related U.S. Application Data
(63) Continuation of application No. 10/892,499, filed on Jul. 16, 2004, now abandoned.
Application No. 10/892,499 is a continuation of application No. 10/665,371, filed on Sep. 22, 2003, now abandoned.
(60) Provisional application No. 60/411,869, filed on Sep. 20, 2002.

(51) Int. Cl. A61K 38/16; A61P 25/00; A61P 9/00

(52) U.S. Cl. 514/13

(57) ABSTRACT

The present invention is directed to kappaM (κM) conopeptide RIIIK and its use for blocking the flow of potassium ions through voltage-gated potassium channels. The κM conopeptides include unglycosylated and O-glycosylated peptides.

### CROSS-REFERENCE TO RELATED APPLICATION

The present application is a continuation of U.S. patent application Ser. No. 10/892,499 filed 16 Jul. 2004, which in turn is a continuation of U.S. patent application Ser. No. 10/665,731 filed 22 Sep. 2003. U.S. patent application Ser. No. 10/665,731 is related to and claims priority under 35 USC § 119(e) to U.S. provisional patent application Ser. No. 60/411,869 filed on 20 Sep. 2002. Each application is incorporated herein by reference.

This invention was made with Government support under Grant No. GM-48677 awarded by the National Institutes of Health, Bethesda, Md. The United States Government has certain rights in the invention.

### BACKGROUND OF THE INVENTION

The present invention is directed to kappaM (κM) conopeptide RIIIK and its use for blocking the flow of potassium ions through voltage-gated potassium channels.

The publications and other materials used herein to illuminate the background of the invention, and in particular, cases to provide additional details respecting the practice, are incorporated by reference, and for convenience are numerically referenced in the following text and respectively grouped in the appended bibliography.

Mollusks of the genus Conus produce a venom that enables them to carry out their unique predatory lifestyle. Prey are immobilized by the venom that is injected by means of a highly specialized venom apparatus, a disposable hollow tooth that functions both in the manner of a harpoon and a hypodermic needle.

Few interactions between organisms are more striking than those between a venomous animal and its envenomated victim. Venom may be used as a primary weapon to capture prey or as a defense mechanism. Many of these venoms contain molecules directed to receptors and ion channels of neuromuscular systems.

The predatory cone snails (Conus) have developed a unique biological strategy. Their venom contains relatively small peptides that are targeted to various neuromuscular receptors and may be equivalent in their pharmacological diversity to the alkaloids of plants or secondary metabolites of microorganisms. Many of these peptides are among the smallest nucleic acid-encoded translation products having defined conformations, and as such, they are somewhat unusual. Peptides in this size range normally equilibrate among many conformations. Proteins having a fixed conformation are generally much larger.

The cone snails that produce these toxic peptides, which are generally referred to as conotoxins or conotoxin peptides, are a large genus of venomous gastropods comprising approximately 500 species. All cone snail species are predators that inject venom to capture prey, and the spectrum of animals that the genus as a whole can envenomate is broad. A wide variety of hunting strategies are used, however, every Conus species uses fundamentally the same basic pattern of envenomation.

Several peptides isolated from Conus venoms have been characterized. These include the α-, μ- and ω-conotoxins which target nicotinic acetylcholine receptors, muscle sodium channels, and neuronal calcium channels, respectively (Olivera et al., 1985). Conopressins, which are vasopressin analogs, have also been identified (Cruz et al., 1987). In addition, peptides named conantokins have been isolated as well (Mena et al., 1990; Haack et al., 1990). These peptides have unusual age-dependent physiological effects: they induce a sleep-like state in mice younger than two weeks and hyperactive behavior in mice older than 3 weeks (Haack et al., 1990). The isolation, structure and activity of κ-conotoxins (now named κA conotoxins) are described in U.S. Pat. No. 5,633,347. Recently, peptides named contryphans containing D-tryptophan residues have been isolated from Conus radiatus (U.S. Pat. No. 6,077,934), and bromo-tryptophan conopeptides have been isolated from Conus imperialis and Conus radiatus (U.S. Pat. No. 5,889,147).

Potassium channels comprise a large and diverse group of proteins that, through maintenance of the cellular membrane potential, are fundamental in normal biological function. These channels are vital in controlling the resting membrane potential in excitable cells and can be broadly sub-divided into three classes: voltage-gated K⁺ channels, Ca²⁺ activated K⁺ channels and ATP-sensitive K⁺ channels. Many disorders are associated with abnormal flow of potassium ions through these channels. The identification of agents which would regulate the flow of potassium ions through each of these channel types would be useful in treating disorders associated with such abnormal flow.

It is desired to identify additional conotoxin peptides having activities of the above conopeptides, as well as conotoxin peptides having additional activities.

### SUMMARY OF THE INVENTION

The present invention is directed to kappaM (κM) conopeptide RIIIK and its use for blocking the flow of potassium ions through voltage-gated potassium channels. The κM conopeptide described herein are useful for treating various disorders as described in further detail herein.

In one embodiment, the present invention is directed to uses of the κM conopeptides described herein for regulating the flow of potassium ions through K⁺ channels. Disorders which can be treated using these conopeptides include multiple sclerosis, other demyelinating diseases (such as acute dissenmiated encephalomyelitis, optic neuromyelitis, adrenoleukodystrophy, acute transverse myelitis, progressive multifocal leukoencephalopathy), sub-acute sclerosing panencephalomyelitis (SSPE), metachromatic leukodystrophy, Pelizaeus-Merzbacher disease, spinal cord injury, botulinum toxin poisoning, Huntington's chorea, compression and entrapment neurophathies (such as carpal tunnel syndrome, ulnar nerve palsy), cardiovascular disorders (such as cardiac arrhythmias, congestive heart failure), reactive gliosis, hyperglycemia, immunosuppression, cocaine addiction, cancer, cognitive dysfunction, disorders resulting from defects in neurotransmitter release (such as Eaton-Lambert syndrome), and reversal of the actions of curare and other neuromuscular blocking drugs. The κM conopeptide RIIIK has the following formula:

Leu-X1-Ser-Cys-Cys-Ser-Leu-Asn-Leu-Arg-Leu-Cys-X1-Val-X1-Ala-Cys-Lys-Arg-Asn-X1-Cys-Cys-Thr (SEQ ID NO:1), wherein X1 is Pro or hydroxy-Pro and the C-terminus is amidated or a free carboxyl.

The present invention is further directed to derivatives of the κM RIIIK described herein or pharmaceutically acceptable salts of these peptides. Substitutions of one amino acid for another can be made at one or more additional sites within the described peptides, and may be made to modulate one or more of the properties of the peptides. Substitutions of this kind are preferably conservative, i.e., one amino acid is replaced with one of similar shape and charge. Conservative substitutions are well known in the art and include, for example: alanine to glycine, arginine to lysine, asparagine to glutamine or histidine, glycine to proline, leucine to valine or isoleucine, serine to threonine, phenylalanine to tyrosine, and the like.

These derivatives also include peptides in which the Pro residues may be substituted by hydroxy-Pro (Hyp); the Glu residues may be substituted by γ-carboxyglutamate (Gla); the Arg residues may be substituted by Lys, ornithine, homoargine, nor-Lys, N-methyl-Lys, N,N-dimethyl-Lys, N,N,N-trimethyl-Lys or any synthetic basic amino acid; the Lys residues may be substituted by Arg, ornithine, homoargine, nor-Lys, N-methyl-Lys, N,N-dimethyl-Lys, N,N,N-trimethyl-Lys or any synthetic basic amino acid; the Tyr residues may be substituted with meta-Tyr, ortho-Tyr, nor-Tyr, mono-halo-Tyr, di-halo-Tyr, O-sulpho-Tyr, O-phospho-Tyr, nitro-Tyr or any synthetic hydroxy containing amino acid; the Ser residues may be substituted with Thr or any synthetic hydroxylated amino acid; the Thr residues may be substituted with Ser or any synthetic hydroxylated amino acid; the Phe residues may be substituted with any synthetic aromatic amino acid; the Trp residues may be substituted with Trp (D), neo-Trp, halo-Trp (D or L) or any aromatic synthetic amino acid; and the Asn, Ser, Thr or Hyp residues may be glycosylated. The halogen may be iodo, radioiodo, chloro, fluoro or bromo; preferably iodo for halogen substituted-Tyr and bromo for halogen-substituted Trp. The Tyr residues may also be substituted with the 3-hydroxyl or 2-hydroxyl isomers (meta-Tyr or ortho-Tyr, respectively) and corresponding O-sulpho- and O-phospho-derivatives. The acidic amino acid residues may be substituted with any synthetic acidic amino acid, e.g., tetrazolyl derivatives of Gly and Ala. The Met residues may be substituted with norleucine (Nle). The aliphatic amino acids may be substituted by synthetic derivatives bearing non-natural aliphatic branched or linear side chains CₙH₂ₙ₊₂ up to and including n=8. The Leu residues may be substituted with Leu (D). The Asn residues may be substituted with Gln. The Gla residues may be substituted with Glu. The N-terminal Gln residues may be substituted with pyroGlu.

The present invention is further directed to derivatives of the above peptides and peptide derivatives which are acylic permutations in which the cyclic permutants retain the native bridging pattern of native toxin. See Craik et al. (2001).

Examples of synthetic aromatic amino acid include, but are not limited to, nitro-Phe, 4-substituted-Phe wherein the substituent is C₁-C₃ alkyl, carboxyl, hyrdroxymethyl, sulphomethyl, halo, phenyl, —CHO, —CN, —SO₃H and —NHAc. Examples of synthetic hydroxy containing amino acid, include, but are not limited to, such as 4-hydroxymethyl-Phe, 4-hydroxyphenyl-Gly, 2,6-dimethyl-Tyr and 5-amino-Tyr. Examples of synthetic basic amino acids include, but are not limited to, N-1-(2-pyrazolinyl)-Arg, 2-(4-piperinyl)-Gly, 2-(4-piperinyl)-Ala, 2-[3-(2S)pyrrolininyl)-Gly and 2-[3-(2S)pyrrolininyl)-Ala. These and other synthetic basic amino acids, synthetic hydroxy containing amino acids or synthetic aromatic amino acids are described in Building Block Index, Version 3.0 (1999 Catalog, pages 4-47 for hydroxy containing amino acids and aromatic amino acids and pages 66-87 for basic amino acids; see also their online catalog), incorporated herein by reference, by and available from RSP Amino Acid Analogues, Inc., Worcester, Ma. Examples of synthetic acid amino acids include those derivatives bearing acidic functionality, including carboxyl, phosphate, sulfonate and synthetic tetrazolyl derivatives such as described by Ornstein et al. (1993) and in U.S. Pat. No. 5,331,001, each incorporated herein by reference, and such as shown in U.S. published patent application No. US 2003/0170222 A1.

Optionally, in the κM RIIIK of the present invention, the Asn residues may be modified to contain an N-glycan and the Ser, Thr and Hyp residues may be modified to contain an O-glycan (e.g., g-N, g-S, g-T and g-Hyp). In accordance with the present invention, a glycan shall mean any N-, S- or O-linked mono-, di-, tri-, poly- or oligosaccharide that can be attached to any hydroxy, amino or thiol group of natural or modified amino acids by synthetic or enzymatic methodologies known in the art. The monosaccharides making up the glycan can include D-allose, D-altrose, D-glucose, D-mannose, D-gulose, D-idose, D-galactose, D-talose, D-galactosamine, D-glucosamine, D-N-acetyl-glucosamine (GlcNAc), D-N-acetyl-galactosamine (GalNAc), D-fucose or D-arabinose. These saccharides may be structurally modified, e.g., with one or more O-sulfate, O-phosphate, O-acetyl or acidic groups, such as sialic acid, including combinations thereof The gylcan may also include similar polyhydroxy groups, such as D-penicillamine 2,5 and halogenated derivatives thereof or polypropylene glycol derivatives. The glycosidic linkage is beta and 1-4 or 1-3, preferably 1-3. The linkage between the glycan and the amino acid may be alpha or beta, preferably alpha and is 1-.

Core O-glycans have been described by Van de Steen et al. (1998), incorporated herein by reference. Mucin type O-linked oligosaccharides are attached to Ser or Thr (or other hydroxylated residues of the present peptides) by a GalNAc residue. The monosaccharide building blocks and the linkage attached to this first GalNAc residue define the “core glycans,” of which eight have been identified. The type of glycosidic linkage (orientation and connectivities) are defined for each core glycan. Suitable glycans and glycan analogs are described further in U.S. patent application Ser. No. 09/420,797 filed 19 Oct. 1999 (now U.S. Pat. No. 6,369,193) and in International Patent Application No. PCT/US99/24380 filed 19 Oct. 1999 (publication No. WO 00/23092), each incorporated herein by reference. A preferred glycan is Gal(β1→3)GalNAc(α1→).

Optionally, in the peptides of general formula I and the specific peptides described above, pairs of Cys residues may be replaced pairwise with isoteric lactam or ester-thioether replacements, such as Ser/(Glu or Asp), Lys/(Glu or Asp), Cys/(Glu or Asp) or Cys/Ala combinations. Sequential coupling by known methods (Barnay et al., 2000; Hruby et al., 1994; Bitan et al., 1997) allows replacement of native Cys bridges with lactam bridges. Thioether analogs may be readily synthesized using halo-Ala residues commercially available from RSP Amino Acid Analogues. In addition, individual Cys residues may be replaced with homoCys, seleno-Cys or penicillamine, so that disulfide bridges may be formed between Cys-homoCys or Cys-penicillamine, or homoCys-penicllamine and the like.

### BRIEF DESCRIPTION OF THE FIGURES

FIGS. 1A-1C show κM-RIIIK does not block Naᵥ1.4 mediated currents. FIG. 1A shows whole cell currents recorded from an oocyte expressing Na, 1.4 Na⁺ channels evoked by test potentials from −80 to +60 mV in steps of 10 mV. Addition of 10 μM of κM-conotoxin RIIIK results in no effect on the evoked currents (FIG. 1B) which is also demonstrated by the current-voltage relationships (FIG. 1C). The dashed line corresponds to zero current.

FIGS. 2A-2B show κM-conotoxin RIIIK blocks Shaker-mediated currents. FIG. 2A: The top panel shows whole cell currents recorded from an oocyte expressing Shaker K⁺ channels evoked by test potentials to 0, 20 and 40 mV. Addition of 2 μM of κM-conotoxin RIIIK results in a block of the currents (middle panel) which is reversible (lower panel). The dashed line corresponds to zero current. FIG. 2B: Dose response curve for the block by κM-conotoxin at a test potential of 0 mV (n=5).

FIGS. 3A-3B show mutations in the pore do affect the binding of κM-conotoxin RIIIK (FIG. 3B) to Shaker channels compared to control (FIG. 3A). FIG. 3A shows whole cell currents from oocytes expressing the mutated channel of Shaker H4. FIG. 3B: Mutating the phenylalanine to glycine (F425G) results in a channel that is insensitive to 2 μM κM-conotoxin RIIIK (left panel). Mutating the lysine 427 to an aspartate (K427D) results in a channel with increased sensitivity to this toxin (middle panel). Mutating the threonine at position 449 to tyrosine (T449Y) results in a channel that is insensitive to 2 μM κM-conotoxin RIIIK. Voltage steps are as in FIGS. 2A-2B.

FIGS. 4A-4D show that the trout homolog of Shaker channels, TSha1, is blocked by κM-conotoxin RIIIK. FIG. 4A: Whole cell currents recorded from an oocyte expressing TSha1 K⁺ channels evoked by test potentials to 0, 20 and 40 mV. FIGS. 4B-4C: Addition of 1 μM of κM-conotoxin RIIIK almost completely blocks the currents (FIG. 4B) in a reversible manner (FIG. 4C). The dashed line corresponds to zero current. FIG. 4D shows the IV-relationship of the evoked current in the absence and presence of 1 μM κM-conotoxin RIIIK.

FIGS. 5A-5C show the block of κM-conotoxin RIIIK of Shaker-Δ6-46, Shaker-Δ6-46 K427D and TSha1 is state-dependent. FIGS. 5A-5B: whole cell currents recorded from oocytes expressing Shaker-Δ6-46, Shaker-Δ6-46 K427D or TSha1 K⁺ channels evoked by test potentials to 0, 20 and 40 mV under control conditions (FIG. 5A), and after addition of the indicated amount of κM-conotoxin RIIIK (FIG. 5B). FIG. 5C: current ratios obtained for the three test potentials showing a single exponential relaxation of the probability of the channels to be unblocked are obtained by calculating I_{Toxin}/I_{Control}.

### DETAILED DESCRIPTION OF THE PREFERRED EMBODIMENTS

The present invention is directed to κM RIIIK and its uses as described above. Potassium channels comprise a large and diverse group of proteins that, through maintenance of the cellular membrane potential, are fundamental in normal biological function. The therapeutic applications for compounds that regulate the flow of potassium ions through K⁺ channels are far-reaching and include treatments of a wide range of disease and injury states. Disorders which can be treated using these conopeptides include multiple sclerosis, other demyelinating diseases (such as acute dissenmiated encephalomyelitis, optic neuromyelitis, adrenoleukodystrophy, acute transverse myelitis, progressive multifocal leukoencephalopathy), sub-acute sclerosing panencephalomyelitis (SSPE), metachromatic leukodystrophy, Pelizaeus-Merzbacher disease, spinal cord injury, botulinum toxin poisoning, Huntington's chorea, compression and entrapment neurophathies (such as carpal tunnel syndrome, ulnar nerve palsy), cardiovascular disorders (such as cardiac arrhythmias, congestive heart failure), reactive gliosis, hyperglycemia, immunosuppression, cocaine addiction, cancer, cognitive dysfunction, disorders resulting from defects in neurotransmitter release (such as Eaton-Lambert syndrome), and reversal of the actions of curare and other neuromuscular blocking drugs.

The κM RIIIK of the present invention is identified by isolation from Conus venom or by using recombinant DNA techniques by screening cDNA libraries of various Conus species using conventional techniques, such as the use of reverse-transcriptase polymerase chain reaction (RT-PCR) or the use of degenerate probes. Clones which hybridize to degenerate probes are analyzed to identify those which meet minimal size requirements, i.e., clones having approximately 300 nucleotides (for a propeptide), as determined using PCR primers which flank the cDNA cloning sites for the specific cDNA library being examined. These minimal-sized clones and the clones produced by RT-PCR are then sequenced. The sequences are then examined for the presence of a peptide having the characteristics noted above for κM-conopeptides. In this manner, κM-RIIIK, a 24 amino acid peptide that was originally cloned from Conus radiatus (U.S. patent application Ser. No. 09/910,009; PCT Published Application WO 02/07678), The biological activity of the peptides identified by this method is tested as described herein or conventionally in the art.

These peptides are sufficiently small to be chemically synthesized. General chemical syntheses for preparing the foregoing conopeptides peptides are described hereinafter, along with specific chemical synthesis of conopeptides and indications of biological activities of these synthetic products. Various ones of these conopeptides can also be obtained by isolation and purification from specific Conus species using the techniques described in U.S. Pat. Nos. 4,447,356 (Olivera et al., 1984), 5,514,774 (Olivera et al., 1996) and 5,591,821 (Olivera et al., 1997), the disclosures of which are incorporated herein by reference.

Although the conopeptides of the present invention can be obtained by purification from cone snails, because the amounts of conopeptides obtainable from individual snails are very small, the desired substantially pure conopeptides are best practically obtained in commercially valuable amounts by chemical synthesis using solid-phase strategy. For example, the yield from a single cone snail may be about 10 micrograms or less of conopeptide. By “substantially pure” is meant that the peptide is present in the substantial absence of other biological molecules of the same type; it is preferably present in an amount of at least about 85% purity and preferably at least about 95% purity. Chemical synthesis of biologically active conopeptides depends of course upon correct determination of the amino acid sequence. Thus, the conopeptides of the present invention may be isolated, synthesized and/or substantially pure.

The conopeptides can also be produced by recombinant DNA techniques well known in the art. Such techniques are described by Sambrook et al. (1989). The peptides produced in this manner are isolated, reduced if necessary, and oxidized to form the correct disulfide bonds, if present in the final molecule.

One method of forming disulfide bonds in the conopeptides of the present invention is the air oxidation of the linear peptides for prolonged periods under cold room temperatures or at room temperature. This procedure results in the creation of a substantial amount of the bioactive, disulfide-linked peptides. The oxidized peptides are fractionated using reverse-phase high performance liquid chromatography (HPLC) or the like, to separate peptides having different linked configurations. Thereafter, either by comparing these fractions with the elution of the native material or by using a simple assay, the particular fraction having the correct linkage for maximum biological potency is easily determined. It is also found that the linear peptide, or the oxidized product having more than one fraction, can sometimes be used for in vivo administration because the cross-linking and/or rearrangement which occurs in vivo has been found to create the biologically potent conopeptide molecule. However, because of the dilution resulting from the presence of other fractions of less biopotency, a somewhat higher dosage may be required.

The peptides are synthesized by a suitable method, such as by exclusively solid-phase techniques, by partial solid-phase techniques, by fragment condensation or by classical solution couplings.

In conventional solution phase peptide synthesis, the peptide chain can be prepared by a series of coupling reactions in which constituent amino acids are added to the growing peptide chain in the desired sequence. Use of various coupling reagents, e.g., dicyclohexylcarbodiimide or diisopropylcarbonyldimidazole, various active esters, e.g., esters of N-hydroxyphthalimide or N-hydroxy-succinimide, and the various cleavage reagents, to carry out reaction in solution, with subsequent isolation and purification of intermediates, is well known classical peptide methodology. Classical solution synthesis is described in detail in the treatise, “Methoden der Organischen Chemie (Houben-Weyl): Synthese von Peptiden,” (1974). Techniques of exclusively solid-phase synthesis are set forth in the textbook, “Solid-Phase Peptide Synthesis,” (Stewart and Young, 1969), and are exemplified by the disclosure of U.S. Pat. No. 4,105,603 (Vale et al., 1978). The fragment condensation method of synthesis is exemplified in U.S. Pat. No. 3,972,859 (1976). Other available syntheses are exemplified by U.S. Pat. Nos. 3,842,067 (1974) and 3,862,925 (1975). The synthesis of peptides containing y-carboxyglutamic acid residues is exemplified by Rivier et al. (1987), Nishiuchi et al. (1993) and Zhou et al. (1996). Synthesis of conopeptides have been described in U.S. Pat. Nos. 4,447,356 (Olivera et al., 1984), 5,514,774 (Olivera et al., 1996) and 5,591,821 (Olivera et al., 1997).

Common to such chemical syntheses is the protection of the labile side chain groups of the various amino acid moieties with suitable protecting groups which will prevent a chemical reaction from occurring at that site until the group is ultimately removed. Usually also common is the protection of an α-amino group on an amino acid or a fragment while that entity reacts at the carboxyl group, followed by the selective removal of the α-amino protecting group to allow subsequent reaction to take place at that location. Accordingly, it is common that, as a step in such a synthesis, an intermediate compound is produced which includes each of the amino acid residues located in its desired sequence in the peptide chain with appropriate side-chain protecting groups linked to various ones of the residues having labile side chains.

As far as the selection of a side chain amino protecting group is concerned, generally one is chosen which is not removed during deprotection of the α-amino groups during the synthesis. However, for some amino acids, e.g., His, protection is not generally necessary. In selecting a particular side chain protecting group to be used in the synthesis of the peptides, the following general rules are followed: (a) the protecting group preferably retains its protecting properties and is not split off under coupling conditions, (b) the protecting group should be stable under the reaction conditions selected for removing the α-amino protecting group at each step of the synthesis, and (c) the side chain protecting group must be removable, upon the completion of the synthesis containing the desired amino acid sequence, under reaction conditions that will not undesirably alter the peptide chain.

It should be possible to prepare many, or even all, of these peptides using recombinant DNA technology. However, when peptides are not so prepared, they are preferably prepared using the Merrifield solid-phase synthesis, although other equivalent chemical syntheses known in the art can also be used as previously mentioned. Solid-phase synthesis is commenced from the C-terminus of the peptide by coupling a protected α-amino acid to a suitable resin. Such a starting material can be prepared by attaching an α-amino-protected amino acid by an ester linkage to a chloromethylated resin or a hydroxymethyl resin, or by an amide bond to a benzhydrylamine (BHA) resin or paramethylbenzhydrylamine (MBHA) resin. Preparation of the hydroxymethyl resin is described by Bodansky et al. (1966). Chloromethylated resins are commercially available from Bio Rad Laboratories (Richmond, Calif.) and from Lab. Systems, Inc. The preparation of such a resin is described by Stewart and Young (1969). BHA and MBHA resin supports are commercially available, and are generally used when the desired polypeptide being synthesized has an unsubstituted amide at the C-terminus. Thus, solid resin supports may be any of those known in the art, such as one having the formulae —O—CH₂-resin support, —NH BHA resin support, or —NH-MBHA resin support. When the unsubstituted amide is desired, use of a BHA or MBHA resin is preferred, because cleavage directly gives the amide. In case the N-methyl amide is desired, it can be generated from an N-methyl BHA resin. Should other substituted amides be desired, the teaching of U.S. Pat. No. 4,569,967 (Kornreich et al., 1986) can be used, or should still other groups than the free acid be desired at the C-terminus, it may be preferable to synthesize the peptide using classical methods as set forth in the Houben-Weyl text (1974).

The C-terminal amino acid, protected by Boc or Fmoc and by a side-chain protecting group, if appropriate, can be first coupled to a chloromethylated resin according to the procedure set forth in Horiki et al. (1978), using KF in DMF at about 60° C. for 24 hours with stirring, when a peptide having free acid at the C-terminus is to be synthesized. Following the coupling of the BOC-protected amino acid to the resin support, the α-amino protecting group is removed, as by using trifluoroacetic acid (TFA) in methylene chloride or TFA alone. The deprotection is carried out at a temperature between about 0° C. and room temperature. Other standard cleaving reagents, such as HCl in dioxane, and conditions for removal of specific α-amino protecting groups may be used as described in Schroder and Lubke (1965).

After removal of the α-amino-protecting group, the remaining α-amino- and side chain-protected amino acids are coupled step-wise in the desired order to obtain the intermediate compound defined hereinbefore, or as an alternative to adding each amino acid separately in the synthesis, some of them may be coupled to one another prior to addition to the solid phase reactor. Selection of an appropriate coupling reagent is within the skill of the art. Particularly suitable as a coupling reagent is N,N′-dicyclohexylcarbodiimide (DCC, DIC, HBTU, HATU, TBTU in the presence of HoBt or HoAt).

The activating reagents used in the solid phase synthesis of the peptides are well known in the peptide art. Examples of suitable activating reagents are carbodiimides, such as N,N′-diisopropylcarbodiimide and N-ethyl-N′-(3-dimethylaminopropyl)carbodiimide. Other activating reagents and their use in peptide coupling are described by Schroder and Lubke (1965) and Kapoor (1970).

Each protected amino acid or amino acid sequence is introduced into the solid-phase reactor in about a twofold or more excess, and the coupling may be carried out in a medium of dimethylformamide (DMF):CH₂Cl₂ (1:1) or in DMF or CH₂Cl₂ alone. In cases where intermediate coupling occurs, the coupling procedure is repeated before removal of the α-amino protecting group prior to the coupling of the next amino acid. The success of the coupling reaction at each stage of the synthesis, if performed manually, is preferably monitored by the ninhydrin reaction, as described by Kaiser et al. (1970). Coupling reactions can be performed automatically, as on a Beckman 990 automatic synthesizer, using a program such as that reported in Rivier et al. (1978).

After the desired amino acid sequence has been completed, the intermediate peptide can be removed from the resin support by treatment with a reagent, such as liquid hydrogen fluoride or TFA (if using Fmoc chemistry), which not only cleaves the peptide from the resin but also cleaves all remaining side chain protecting groups and also the α-amino protecting group at the N-terminus if it was not previously removed to obtain the peptide in the form of the free acid. If Met is present in the sequence, the Boc protecting group is preferably first removed using trifluoroacetic acid (TFA)/ethanedithiol prior to cleaving the peptide from the resin with HF to eliminate potential S-alkylation. When using hydrogen fluoride or TFA for cleaving, one or more scavengers such as anisole, cresol, dimethyl sulfide and methylethyl sulfide are included in the reaction vessel.

Cyclization of the linear peptide is preferably affected, as opposed to cyclizing the peptide while a part of the peptido-resin, to create bonds between Cys residues. To effect such a disulfide cyclizing linkage, fully protected peptide can be cleaved from a hydroxymethylated resin or a chloromethylated resin support by ammonolysis, as is well known in the art, to yield the fully protected amide intermediate, which is thereafter suitably cyclized and deprotected. Alternatively, deprotection, as well as cleavage of the peptide from the above resins or a benzhydrylamine (BHA) resin or a methylbenzhydrylamine (MBHA), can take place at 0° C. with hydrofluoric acid (HF) or TFA, followed by oxidation as described above. A suitable method for cyclization is the method described by Cartier et al. (1996).

Muteins, analogs or active fragments, of the foregoing κM-conotoxin peptides are also contemplated here. See, e.g., Hammerland et al (1992). Derivative muteins, analogs or active fragments of the conotoxin peptides may be synthesized according to known techniques, including conservative amino acid substitutions, such as outlined in U.S. Pat. Nos. 5,545,723 (see particularly col. 2, line 50 to col. 3, line 8); 5,534,615 (see particularly col. 19, line 45 to col. 22, line 33); and 5,364,769 (see particularly col. 4, line 55 to col. 7, line 26), each incorporated herein by reference.

Pharmaceutical compositions containing a compound of the present invention as the active ingredient can be prepared according to conventional pharmaceutical compounding techniques. See, for example, Remington's Pharmaceutical Sciences, 18th Ed. (1990, Mack Publishing Co., Easton, Pa.). Typically, an antagonistic amount of the active ingredient will be admixed with a pharmaceutically acceptable carrier. The carrier may take a wide variety of forms depending on the form of preparation desired for administration, e.g., intravenous, oral or parenteral. For examples of delivery methods, see U.S. Pat. No. 5,844,077, incorporated herein by reference.

For oral administration, the compounds can be formulated into solid or liquid preparations such as capsules, pills, tablets, lozenges, melts, powders, suspensions or emulsions. In preparing the compositions in oral dosage form, any of the usual pharmaceutical media may be employed, such as, for example, water, glycols, oils, alcohols, flavoring agents, preservatives, coloring agents, suspending agents and the like in the case of oral liquid preparations (such as, for example, suspensions, elixirs and solutions); or carriers such as starches, sugars, diluents, granulating agents, lubricants, binders, disintegrating agents and the like in the case of oral solid preparations (such as, for example, powders, capsules and tablets). Because of their ease in administration, tablets and capsules represent the most advantageous oral dosage unit form, in which case solid pharmaceutical carriers are obviously employed. If desired, tablets may be sugar-coated or enteric-coated by standard techniques. The active agent can be encapsulated to make it stable for passage through the gastrointestinal tract, while at the same time allowing for passage across the blood brain barrier. See for example, WO 96/11698.

For parenteral administration, the compound may be dissolved in a pharmaceutical carrier and administered as either a solution or a suspension. Illustrative of suitable carriers are water, saline, dextrose solutions, fructose solutions, ethanol, or oils of animal, vegetative or synthetic origin. The carrier may also contain other ingredients, for example, preservatives, suspending agents, solubilizing agents, buffers and the like. When the compounds are being administered intrathecally, they may also be dissolved in cerebrospinal fluid.

The active agent is preferably administered in a therapeutically effective amount. The actual amount administered, and the rate and time-course of administration, will depend on the nature and severity of the condition being treated. Prescription of treatment, e.g. decisions on dosage, timing, etc., is within the responsibility of general practitioners or specialists, and typically takes into account the disorder to be treated, the condition of the individual patient, the site of delivery, the method of administration and other factors known to practitioners. Examples of techniques and protocols can be found in Remington's Pharmaceutical Sciences. Typically, the active agents of the present invention exhibit their effect at a dosage range of from about 0.001 mg/kg to about 250 mg/kg, preferably from about 0.01 mg/kg to about 100 mg/kg, of the active ingredient and more preferably, from about 0.05 mg/kg to about 75 mg/kg. A suitable dose can be administered in multiple sub-doses per day. Typically, a dose or sub-dose may contain from about 0.1 mg to about 500 mg of the active ingredient per unit dosage form. A more preferred dosage will contain from about 0.5 mg to about 100 mg of active ingredient per unit dosage form. Dosages are generally initiated at lower levels and increased until desired effects are achieved.

Alternatively, targeting therapies may be used to deliver the active agent more specifically to certain types of cells, by the use of targeting systems such as antibodies or cell-specific ligands. Targeting may be desirable for a variety of reasons, e.g. if the agent is unacceptably toxic, if it would otherwise require too high a dosage, or if it would not otherwise be able to enter target cells.

The active agents, which are peptides, can also be administered in a cell-based delivery system in which a DNA sequence encoding an active agent is introduced into cells designed for implantation in the body of the patient, especially in the spinal cord region. Suitable delivery systems are described in U.S. Pat. No. 5,550,050 and in published PCT Applications No. WO 92/19195, WO 94/25503, WO 95/01203, WO 95/05452, WO 96/02286, WO 96/02646, WO 96/40871, WO 96/40959 and WO 97/12635. Suitable DNA sequences can be prepared synthetically for each active agent on the basis of developed sequences and the known genetic code.

### EXAMPLES

The present invention is described by reference to the following Examples, which are offered by way of illustration and are not intended to limit the invention in any manner. Standard techniques well known in the art or the techniques specifically described below were utilized.

Voltage-gated ion channels determine the membrane excitability of cells. Although many Conus peptides that interact with voltage-gated Na⁺ and Ca⁺⁺ channels have been characterized, relatively few have been identified that interact with K⁺ channels. We describe a novel Conus peptide that interacts with the Shaker K⁺ channel, κM-conotoxin RIIIK from Conus radiatus. The peptide was chemically synthesized. Although κM -Conotoxin RIIIK is structurally similar to the κM -conotoxins that are sodium channel blockers, it does not affect any of the sodium channels tested, but blocks Shaker K⁺ channels.

Studies using Shaker K⁺ channel mutants with single residue substitutions reveal that the peptide interacts with the pore region of the channel. Introduction of a negative charge at residue 427 (K427D) greatly increases the affinity of the toxin, while the substitutions at two other residues, F425 and T449, drastically reduced toxin affinity. Based on the Shaker results, a teleost homolog of the Shaker K⁺ channel, TSha1 was identified as a κM -conotoxin RIIIK target. Binding of κM M-conotoxin RIIIK is state dependent, with an IC₅₀ of 20 nM for the closed state and 60 nM at 0 mV for the open state of TSha1 channels.

### Example 1

### Experimental Procedures

Abbreviations: FMOC, N-(9-fluorenyl)methoxycarboxyl; HPLC; high performance liquid chromatography; i.c.v., intracerbrovascular; i.p., intraperitoneal; i.t, intrathecal; TFA, trifluoroacetic acid.

Synthesis: The peptides were synthesized on Rink amide resin using FMOC chemistry and standard side chain protection except for the cysteine residues. For all the peptides, the cysteine side chains were trityl protected.

Peptides were removed from the resin as described previously (Cartier et al., 1996; Jacobsen et al., 1997). Preparative purification of the linear peptides was carried out by HPLC with either a 5-55% or 10-50% gradient of 0.1% TFA (buffer A) and 0.1% TFA, 60% acetonitrile (buffer B60). The standard one-step oxidation protocol was used to fold the peptides. Fully oxidized peptides were purified by preparative HPLC using either a 5-55% or 10-50% gradient of B60.

Electrophysiological methods: The Xenopus expression system was used for investigating the potential effects of κM-conotoxin RIIIK on voltage-gated Na⁺ and K⁺ channels. Oocytes from Xenopus laevis were prepared as described previously (Methfessel et al., 1986; Stühmer, 1992). Frogs were anaesthetized with 0.2% tricaine in ice water for surgery. Following cRNA injection, the oocytes were incubated 1-5 days to allow expression of the protein. Prior to the electrophysiological measurements, the vitelline membranes of the oocytes were removed mechanically with fine forceps. cRNAs encoding various cloned Na⁺ and K⁺ channels to be tested were prepared by standard techniques. Whole cell currents were recorded under two-electrode voltage clamp control using a Turbo-Tec amplifier (npi electronic, Tamm Germany). The intracellular electrodes were filled with 2 M KCl and had a resistance between 0.6 and 1 MΩ. Current records were low-pass filtered at 1 kHz (K⁺ channels) or 3 kHz (Na⁺ channels) (−3 dB) and sampled at 4 or 10 kHz, respectively. Leak and capacitive currents were corrected online by using a P/n method. The bath solution was normal frog Ringer's (NFR) containing (in mM): 115 NaCl, 2.5 KCl, 1.8 CaCl₂, 10 Hepes pH 7.2 (NaOH). Lyophilized κM-conotoxin RIIIK was dissolved in NFR, diluted to the final concentration and added to the bath chamber. All electrophysiological experiments were performed at room temperature (19-22° C.).

The IC₅₀ values for the block of Shaker wild-type and the Shaker K427D channels were calculated from the peak currents at a test potential of 0 mV according to IC₅₀=fc/(1−fc)*[Tx], where fc is the fractional current and [Tx] is the toxin concentration. For the binding of κ-conotoxin PVIIA to Shaker channels, it was shown that this is an approximation to obtain the affinity of the toxin to the closed state of the channel (see below). Data are given as mean±S.D.

The kinetic parameters of the state-dependent block for the different channels investigated were obtained as described in (Terlau et al., 1999).

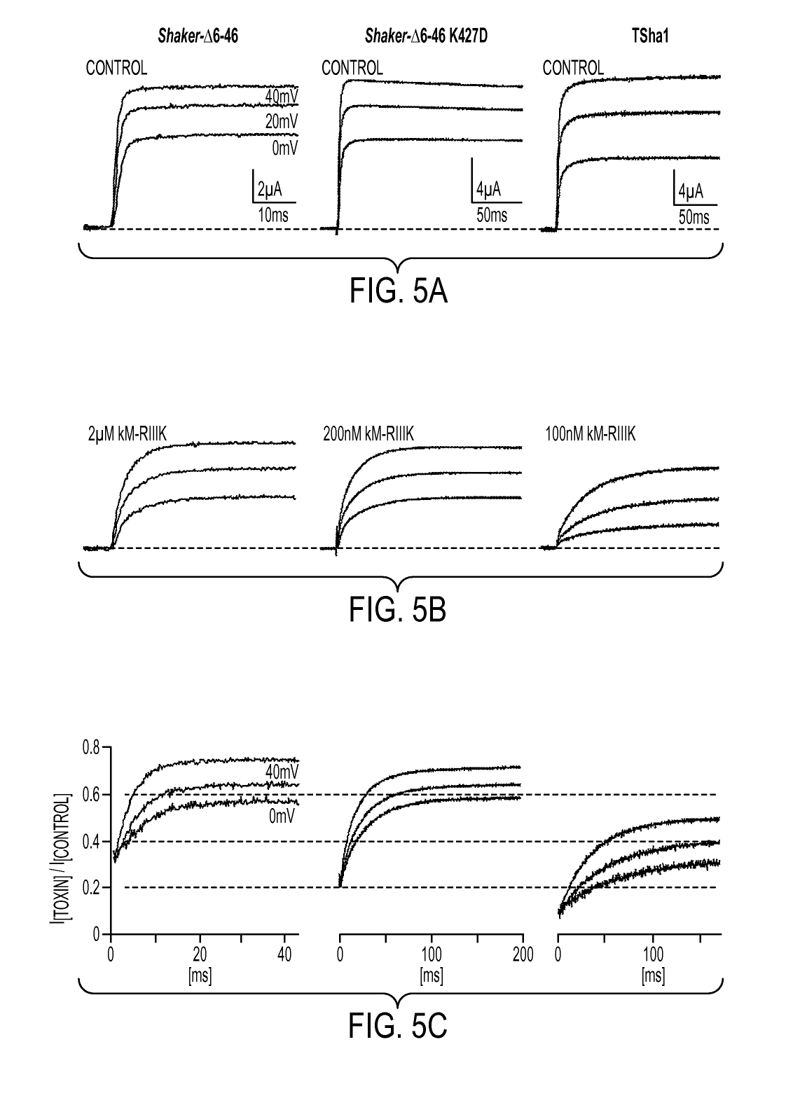

Open channel binding: From the ratio of the currents under control and toxin conditions a single exponential relaxation of the block is observed (see FIGS. 5A-5C) which can be interpreted by a simple bimolecular reaction scheme:

$$\{ U \} {{{k}_{off}}_{}}^{{k}_{on} * [ T ]} \{ B \}$$

where {U} represents the toxin-free channels and {B} the channels bound to a toxin molecule. From the experimental parameters τ and U it is possible to evaluate K^{O}, k_{off}^{O}, kₒₙ^{O} according to the inverse relationships:

$${K}^{( O )} = \frac{[ T ] * {U}^{( O )}}{1 - {U}^{( O )}} ;$$

$${k}_{off}^{( O )} = \frac{{U}^{( O )}}{{τ}^{( O )}} ;$$

$${k}_{on}^{( O )} = \frac{1 - {U}^{( O )}}{[ T ] * {τ}^{( O )}} .$$

Closed channel binding: The parameters of toxin binding to the closed state were obtained by performing a similar analysis for the currents obtained from double pulse protocols (16).

Molecular biology: The cDNA library from C. radiatus venom ducts was prepared as previously described (Colledge et al., 1992; Jacobsen et al., 1998). The wild-type, Δ6-46 and substitution mutant clones of the Shaker K⁺ channel were a generous gift of Dr. Martin Stocker.

### Example 2

### Cloning and Synthesis of the κM-Conotoxin

A cDNA clone from a Conus radiatus venom duct library was sequenced and the predicted mature peptide sequence deduced from rules now standard for conopeptide precursors. The predicted sequence, one including the expected post-translational processing of the original ribosomally-translated polypeptide, is shown in Table 1. Because in all known μ-conotoxins and ψ-conotoxins (the groups with amino acid sequences most related to that of the of the mature κM peptide), proline residues are always found to be hydroxylated, the peptide is inferred to have a hydroxyproline residue at all loci encoded by a proline codon. The sequence N-terminal to the mature toxin contains a canonical dibasic signal for proteolytic cleavage (underlined in Table I) while the C-terminal amino acid sequence predicted by the clone would be expected to be post-translationally processed to an amidated C-terminal threonine residue.

TABLE I
Nucleic Acid Sequence of cDNA Clone and Predicted Processing of Peptide

--- GAA AAG AGA CTA CCA TCG TGT TGC TCC CTT AAC TTG CGG CTT TGC
--- E K R L P S C C S L N L R L C
CCA GTA CCA GCA TGC AAA CGT AAC CCT TGT TGC ACA GGA TAA ---(SEQ ID NO:2
P V P A C K R N P C C T G * ---(SEQ ID NO:3)

---EKRLOSCCSLNLRLCOVOACKRNOCCTG (SEQ ID NO3)
(C-terminus of precursor)

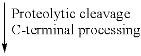

LOSCCSLNLRLCOVOACKRNOCCT-NH₂ (SEQ ID NO:1)
(Predicted mature peptide)

O = 4-trans-hydroxyproline.

The predicted mature peptide that was chemically synthesized is shown in the bottom of Table I. Procedures used in the chemical synthesis and folding of the peptide are detailed under Experimental Procedures.

### Example 3

### Biological Activity and Electrophysiological Characterization

The peptide elicited obvious symptomatology upon injection into mice both i.c.v. and i.t. When 4 nmoles of the synthetic peptide were injected by the i.c.v. route, seizures were observed. However, when the peptide was injected i.p. into mice, there were no visible effects. Electrophysiological experiments using amphibian nerve-muscle preparations were similarly unaffected by 10 μM of the peptide.

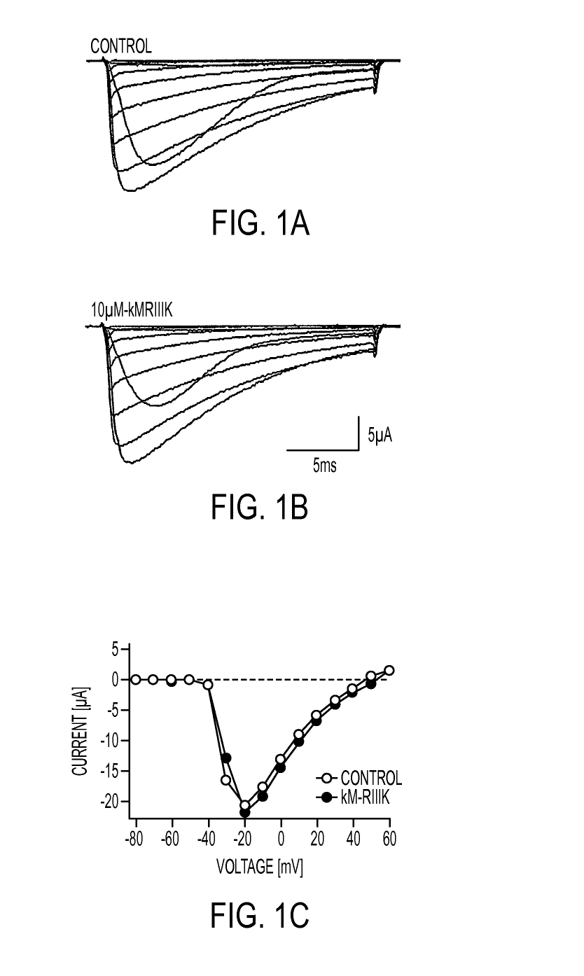

Because the peptide has a Class III framework similar to the μ-conotoxins (which are Na channel ligands), the effects of the synthetic κM peptide on three cloned Na⁺ channel subtypes (i.e., Naᵥ1.2 (rat brain Type II), Naᵥ1.4 (rat skeletal muscle) and Naᵥ1.5 (mouse cardiac channel)) subtypes} expressed in Xenopus oocyte were examined (see Experimental Procedures). At a concentration of 2 μM or 10 μM, the peptide did not show any detectable effect on the currents produced by these cloned Na channel subtypes. This is shown in FIGS. 1A-1C for Naᵥ1.4 which is a high affinity target of μ-conotoxins GIIIA and PIIIA.

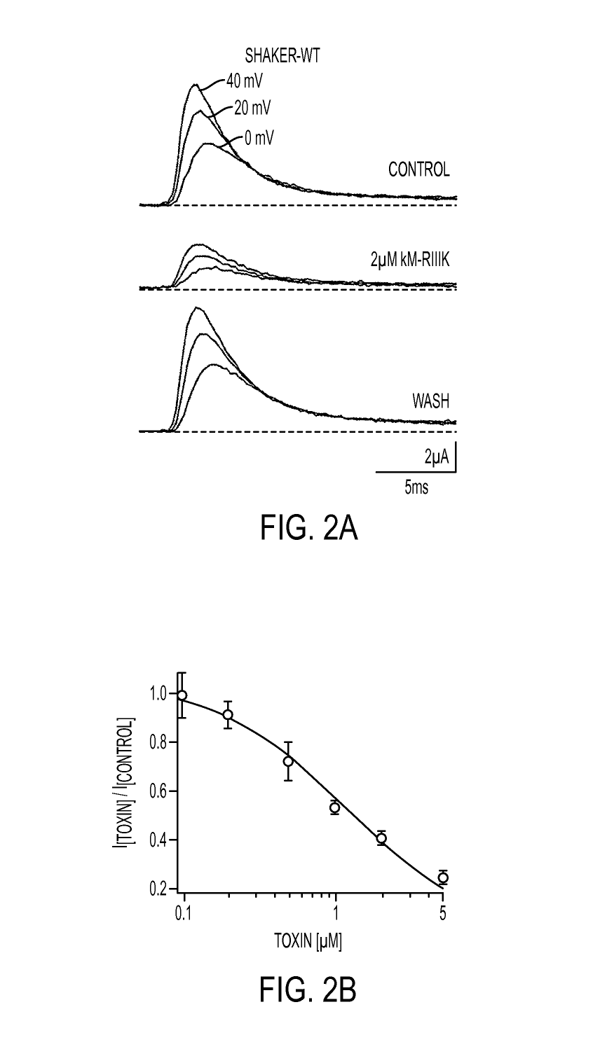

The peptide has been tested on nine different cloned potassium channels. No activity (with 10 μM peptide) was detected on Kv1.1, Kv1.3, Kv1.4, Kv2.1, Kv3.4, Kv4.2, herg and reag K⁺ channel clones expressed in oocytes. However, when the peptide was tested on the Shaker K⁺ channel, an inhibition of channel conductance was observed as shown in FIGS. 2A-2B. The inhibition is readily reversible as shown in the bottom panel of FIG. 2A. The IC₅₀ for the Shaker channel obtained from measuring the peak currents is 1.21±0.25 μM (n=5 dose response experiments—see FIG. 2B). The Hill coefficient is ˜1, suggesting that binding of a single toxin molecule is sufficient to inhibit the Shaker channel.

### Example 4

### Studies using Shaker K⁺ Channel Mutants with Single Residue Subsitutions

The interaction of C. radiatus peptide with a number of mutants of the Shaker potassium channel were assessed. Many ligands that decrease the conductance of the Shaker K⁺ channel bind to the outer vestibule of the Shaker channel. Among the key amino acids in this general region found to affect the affinity of other Shaker K⁺ channel ligands are F425, K427 and T449. We therefore determined whether single amino acid substitutions at these loci might affect the affinity of the C. radiatus peptide for the Shaker channel.

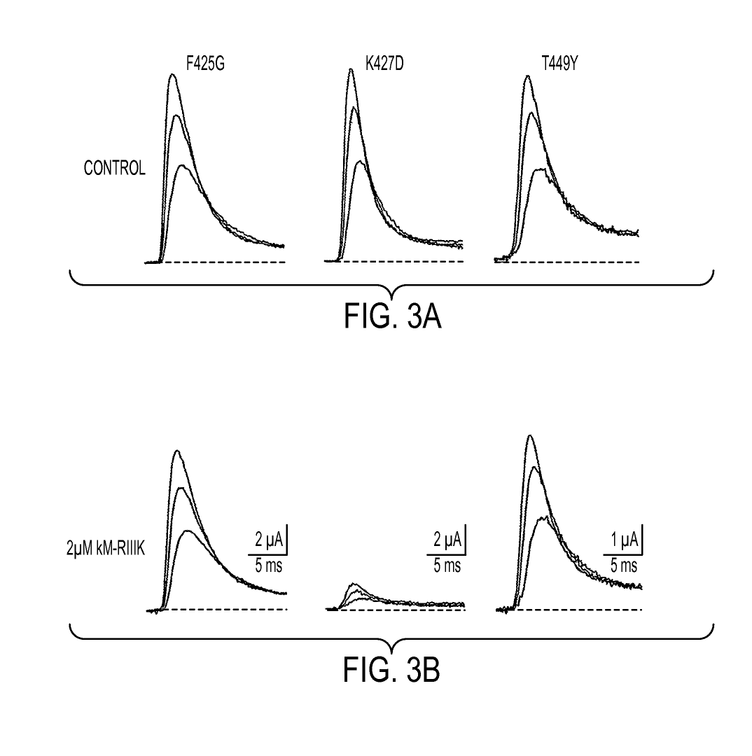

The results for three different amino acid substitutions (F425G, K427D and T449Y) are shown in FIGS. 3A-3B. The different substitutions show strikingly different effects; two of the mutant channels, F425G and T449Y (the latter affects the TEA sensitivity of the Shaker channel) were found to be much more resistant to the C. radiatus peptide. In contrast, the K427D mutant exhibited about a 10-fold greater affinity for the toxin (IC₅₀=109±61 nM, n=5) than was observed for the wild-type Shaker channel. A smaller increase is observed when K427 is substituted with a neutral amino acid leading to an IC₅₀ of 180±27 nM (n=3) for the K427N substitution. The results in FIGS. 3A-3B reveal that substitution of any of the three residues, believed to be near the extracellular opening of the channel pore, significantly affects toxin affinity. Thus, the three AAs appear to be significant determinants for the peptide to bind to the Shaker K⁺ channel, with either increases or decreases in affinity observed.

The data are consistent with the C. radiatus peptide blocking the conductance of the Shaker K⁺ channel by interactions with the outer vestibule region. Presumably, toxin binding would block Shaker channel conductance by impeding transit of K⁺ through the extracellular opening of the pore.

### Example 5

### Effects on a Fish K⁺ Channel: the Sha1 K⁺ Channel from Trout

Since Conus radiatus is believed to be a fish-hunting cone snail, the results above suggest that the presumptive physiologically-relevant molecular target is a voltage-gated K⁺ channel in fish. We tested one teleost K⁺ channel available as a cDNA clone, the Sha1 channel from trout (Nguyen et al., 2000). We chose the Sha1 channel because the results with Shaker showed that the K427D Shaker mutant has a higher affinity than wild-type, and the in the trout sequence, the homologous position to K427 in Shaker has a Glu residue (see Table II).

TABLE II

Comparison of Shaker and TSha1 K⁺ Channel
Sequences

| | | | | |
|---|---|---|---|---|
| Shaker | S5 | EAGSENSFFKSIPDAFWWAVVTMT | S6 | (SEQ ID NO:4) |
| | | TVGYGDMTPVGVWGK | | |
| Tsha1 | S5 | EADEPESQFESIPDAFWWAVVSMT | 36 | (SEQ ID NO:5) |
| | | TVGYGDMVPTTIGGK | | |

Shown is the TSha1 AA sequence in the P-loop region between S5 and S6 (amino acids 345-383; Nguyen et al., 2000) aligned with the corresponding Shaker sequence (amino acids 48-456).

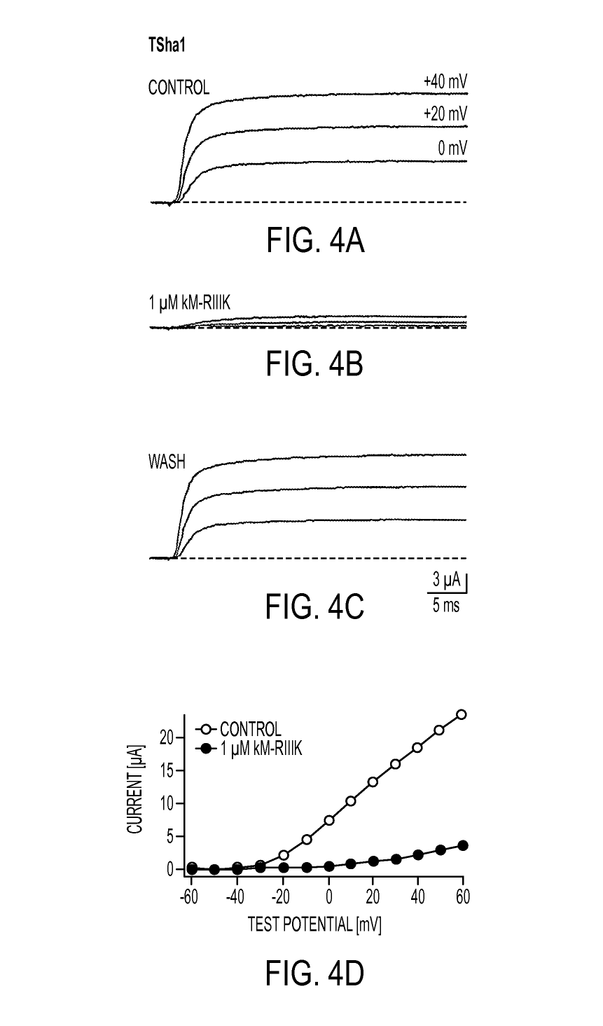

The results are shown in FIGS. 4A-4D. The Sha1 channel, which is a noninactivating voltage-gated K⁺ channel, is more potently inhibited by the Conus radiatus peptide than is the Shaker channel. At a concentration of 1 μM of κM-conotoxin RIIIK the evoked currents are almost completely inhibited. The inhibition is reversible, as well as voltage-dependent. The effects of the toxin as a function of test potential are shown in the FIG. 4D. These results directly establish that the toxin is able to block the conductance of a vertebrate voltage-gated K⁺ channel.

### Example 6

### State Dependence of Shaker K⁺ Channel Inhibition

We evaluated whether the affinity of the peptide changed as a function of the state of the Shaker channel. The open channel properties were investigated by relaxation of partial block during step depolarizations. For this work, Δ6-46 channels of Shaker lacking N-type inactivation were used: this made it easier to evaluate unblocking of open channels. A sample of results obtained are shown in FIG. 5; the unblock follows an exponential time course and is voltage dependent. Similar results were obtained for Shaker-Δ6-46 K427D and the TSha1 (FIGS. 5A-5C).

Double-pulse protocols were used to characterize the re-equilibration of closed channel binding (see Terlau et al., 1999). The data derived from the two types of experiments were used to calculate kinetic parameters (Kₒₙ, K_{off} and IC₅₀) for both the open and the closed states of the Shaker-Δ6-46 channel, Shaker-Δ6-46 K427D and the TSha1 from trout (see Table III). The calculations demonstrate that binding of this toxin to open vs. closed channels is very different, i.e., that the toxin interactions with the Shaker channel are state dependent.

TABLE III
Summary of K_{D},
kₒₙ and k_{off} values

| | OPEN STATE | | | | CLOSED STATE | | | |
|---|---|---|---|---|---|---|---|---|
| | k^{(O)} [0 mV] | k^{(O)}ₒₙ | k^{(O)}_{off} | | k^{(C)} | k^{(C)}ₒₙ | k^{(C)}_{off} | |
| | nM | μM⁻¹*s⁻¹ | s⁻¹ | n | nM | μM⁻¹*s⁻¹ | s⁻¹ | n |
| Shaker -Δ6-46 | 3330 ± 1960 | 30 ± 7 | 90 ± 30 | 3 | 1260 ± 360 | 4.0 ± 0.7 | 6.0 ± 3.0 | 4 |
| Shaker -Δ6-46 | 392 ± 194 | 57 ± 26 | 19 ± 2.5 | 3 | 146 ± 54 | 16.7 ± 8.6 | 2.2 ± 0.9 | 6 |
| K427D | | | | | | | | |
| Tsha 1 | 65 ± 31 | 88 ± 25 | 5.2 ± 1.8 | 4 | 18 ± 12 | 16.9 ± 6.9 | 0.25 ± 0.04 | 3 |

For all three channel types the affinity of the toxin for the open state measured at 0 mV is about three to four times lower compared to the closed state. The results show that the affinity of κM-conotoxin RIIIK to Tsha1 from trout is about 20 nM for the closed and 60 nM for the open state measured at 0 mV. This demonstrates that Tsha1 is a high affinity target for this peptide. The other data are in accordance with the results obtained with Shaker wild type and K427D channels (with inactivation; FIGS. 2A-2B and 3A-3B) demonstrating that for fast inactivating channels the calculation of the IC₅₀ from the peak currents is an approximation for the affinity to the closed state of those channels (see Terlau et al., 1999).

Furthermore, Table III shows that the major reason for the lower affinities of the □M-peptide to the open state of the channels is an increase in k^{(O)}_{off} but also the k^{(O)}ₒₙ, is affected. The comparison of the kinetic parameters of the binding for the three different channel reveals that the k^{(O)}_{off} for open channel binding for Tsha1 is about 20 times smaller than in the Shaker-Δ6-46 channel. In contrast the k^{(O)}ₒₙ in Tsha1 is only three times higher than in the Shaker-Δ6-46 channel. The binding kinetics of κM-RIIIK to the closed state of the three channel types investigated is slower than the binding to the open state.

The data shown in Table III indicate that the negatively-charged residues in Shaker-Δ6-46 K427D and the TSha1 channel strongly affect k^{(C)}ₒₙ, which is almost identical in these two channels, but about four times higher than in the Shaker-Δ6-46 channel. In contrast, the k^{(C)}_{off} differs by a factor of three between Shaker-Δ6-46 and Shaker-Δ6-46 K427D channel, but by a factor of 10 between the Shaker-Δ6-46 K427D channel and Tsha1. This indicates that a negatively-charged AA at residue 427 is a key determinant for rapid association, while k^{(C)}_{off} is primarily influenced by other residues.

The peptide characterized above, though structurally related to the μ-conotoxins that block voltage-gated Na⁺ channel conductance, clearly inhibits the Shaker K⁺ channel. We designate this peptide as κM-conotoxin RIIIK, the first member of a new family of conotoxins targeted to K⁺ channels.

The characterization of κM-RIIIK is noteworthy in several respects. First, the discovery that κM-RIIIK targets the Shaker potassium channel provides a new scaffold for a Shaker channel ligand, the smallest disulfide-crosslinked framework so far characterized for any polypeptide antagonist of K⁺ channels. Because this is the most intensively studied voltage-gated ion channel both from a molecular and functional perspective, the availability of a novel framework in a small polypeptide ligand provides a new platform for examining the topology of this and related channels. We have also established that the interaction of κM-conotoxin RIIIK with the Shaker channel is state dependent. Thus, the peptide is a potentially useful probe for conformational changes that occur in the transition of a voltage-gated ion channel from a closed to an open state.

It was unexpected that a scaffold well known for sodium channel-targeted ligands would also be used by a Conus species to target potassium channels. In retrospect, since these ion channels belong to the same superfamily, the observation can be rationalized ex post facto. As will be detailed elsewhere, the μ-conotoxins and κM-conotoxin RIIIK belong to the same gene superfamily of conopeptides.

The sequences of two μ-conotoxins and a ψ-conotoxin are compared to the κM-conotoxin in Table IV. The κM-conotoxin is distinctive in having a longer first loop, between second and third cysteine residues. Structure/function studies on the μ-conotoxins have established that the arginine residue indicated by the arrow (Arg13 in μ-conotoxin GIIIA) is a critical residue for blocking voltage-gated sodium channels, since the charged guanidino group of the arginine residue is believed to functionally block the pore. In contrast, both the ψ-conotoxins and κM-conotoxin RIIIK lack this critical arginine residue. These differential biochemical characteristics provide a guide in the search for, and identification of, additional members of the κM-conotoxin family. Considerable work has been done on μ-conotoxin:Na channel interactions. Whether the orientation of κM-conotoxin within the potassium channel is analogous to the orientation of μ-conotoxins in the outer vestibule region of sodium channels remains to be determined.

TABLE IV

Comparison of κM-conotoxin RIIIK to Other
M-Superfamily Peptides and κ-PVIIA

| | | |
|---|---|---|
| κM-RIIIK | LOSCCSLNLRLCOVOACKRNOCCT# | (SEQ ID NO:1) |
| μ-GIIA | RDCCTOOKK-CKDRQCKOQRCCA# | (SEQ ID NO:6) |
| μ-PIIIA | ZRCCGFOKS-CRSRQCKOHRCC# | (SEQ ID NO:7) |
| | ↑ | |
| ψ-PIIIE | HOCCLYGKCRRYOGCSSASCCQR# | (SEQ ID NO:8) |
| κ-PVIIA | CRIONQKCFQHLDDCCSRKCNRFNKCV | (SEQ ID NO:9) |

O = 4-trans-hydroxyproline; Z = pyroglutamate, # denotes an amidated C-terminal amino acid. The arrow indicates the Arg residue known to be critical for μ-conotoxin function, which is absent in κM-RIIIK. Note that the κM-, μ- and ψ-conotoxins all have the same pattern of Cys residues, while κ-PVIIA, the only other peptide that also blocks K+ channels, has an entirely different arrangement of Cys.

κM-Conotoxin RIIIK is not the only conopeptide known to inhibit the Shaker K⁺ channel by binding to the outer vestibule. This was first demonstrated for κ-conotoxin PVIIA from Conus purpurascens (Terlau et al., 1996; Shon et al., 1998; Jacobsen et al., 2000). Although both C. radiatus (the source of κM-RIIIK) and C. purpurascens are probably fish-hunting, they are not closely related species as judged by available molecular phylogeny data for the genus Conus (Espiritu et al., 2001; Duda et al., 2001). Because of the accelerated evolution of venom peptides during speciation through focal mutaton (Olivera et al., 1990; Olivera et al., 1997; Woodward et al., 1990; Olivera et al. 1999; Bulaj et al., 2001), the different clades of Conus species use a different spectrum of conotoxin families as major ligands in their venoms. The results presented here establish that two different species of cone snails have evolved structurally and genetically unrelated peptides, both of which block the Shaker K⁺ channel. The two peptides have entirely different structural scaffolds—κM-RIIIK is most closely related to the μ-conotoxins, while κ-PVIIA has the greatest structural similarity to the ω-conotoxins, which target voltage-gated Ca⁺² channels. Even more conopeptides have been found that target the Shaker K⁺ channel, and which are genetically and structurally unrelated to either PVIIA or κM-RIIIK. Thus, screening a broad range of Conus venoms makes it possible to identify a structurally diverse set of ligands that target a given ion channel subtype.

The effect of Shaker K⁺ channel amino acid substitutions on the interaction with κM-conotoxin RIIIK has provided insight into the physiologically relevant channel target of the peptide. The discovery that the K427D Shaker mutant had a higher affinity for the toxin suggested that the physiological target of this peptide might have a negatively-charged residue at the homologous locus. Since Conus radiatus is believed to be a piscivorous Conus species, we examined the sequences of recently cloned teleost channels (Nguyen et al., 2000) related to the Shaker potassium channel. Because the Sha1 channel from trout had a negative residue at this position, we tested the peptide on the trout channel expressed in oocytes. The trout Sha1 channel was potently inhibited by κM-conotoxin RIIIK with an IC₅₀ of 20 nM for the closed state. Thus, this teleost voltage-gated K⁺ channel subtype is a better target for κM-conotoxin RIIIK than Shaker, exhibiting a an almost 50 fold higher affinity. Although little is known about the true teleost prey of Conus radiatus, we postulate that the actual high affinity target of κM-conotoxin RIIIK is a voltage-gated K⁺ channel related to the trout Sha1 channel. However, the full spectrum of K⁺ channels in teleost fish has not yet been elucidated.

Previously, it has been suggested that positively-charged toxins have an accelerated dissociation from open channels due to the voltage-dependent occupancy of a site at the outer end of the conducting pore by a K⁺ ion (Terlau et al., 1999). The site has been postulated to also be occupied by external cations in closed channels, thereby antagonizing the association rate. Our results with the Conus radiatus peptide, which is positively charged, are generally consistent with this model and suggest that electrostatic interactions between the peptide and K⁺ ions in the pore are a major factor in state-dependent binding.

It will be appreciated that the methods and compositions of the instant invention can be incorporated in the form of a variety of embodiments, only a few of which are disclosed herein. It will be apparent to the artisan that other embodiments exist and do not depart from the spirit of the invention. Thus, the described embodiments are illustrative and should not be construed as restrictive.

### BIBLIOGRAPHY

Barber, M. et al. (1982). Anal Chem. 54:645A-657A.

Barnay, G. et al. (2000). J. Med. Chem.

Bitan, G. et al. (1997). J. Peptide Res. 49:421-426.

Bodansky et al. (1966). Chem. Ind. 38:1597-98.

Bulaj, G. et al. (2001). Biochemistry 40:13201-13208.

Cartier, G. E. et al. (1996). J. Biol. Chem. 271:7522-7528.

Colledge, C. J. et al. (1992). Toxicon 30:1111-1116.

Craik, D. J. et al. (1991). Toxicon 39:43-60.

Cruz, L. J. et al. (1987). J. Biol. Chem. 260:9280-9288.

Dodd, J. et al. (1983). J. Physiol. 334:255-268.

Duda Jr, T. F. et al. (2001). Biol. J. Linnean Soc. 73:391-409.

Espiritu, D. J. D. et al. (2001). Toxicon 39:1899-1916.

Gobbo, M. et al. (1992). Int. J. Pept. Protein Res. 40:51-54.

Haack, J. A. et al. (1990). J. Biol. Chem. 265:6025-6029.

Hammerland, L. G. et al. (1992). Eur. J. Pharmacol. 226:239-244.

Horiki, K. et al. (1978). Chemistry Letters 165-68.

Hubry, V. et al. (1994). Reactive Polymers 22:231-241.

Jacobsen, R. et al., (1997). J. Biol. Chem. 272:22531-22537.

Jacobsen, R. et al. (1998). J. Peptide Res. 51:173-179.

Jacobsen, R. B. et al. (2000). J. Biol. Chem. 275:24639-24644.

Kaiser et al. (1970). Anal. Biochem. 34:595.

Kapoor (1970). J. Pharm. Sci. 59:1-27.

Lowry, O. et al. (1951). J. Biol. Chem. 193:265.

Mena, E. E. et al. (1990). Neurosci. Lett. 118:241-244.

Methfessel, C. et al. (1986). Pflugers Arch. 407:577-588.

Methoden der Organischen Chemie (Houben-Weyl): Synthese von Peptiden, E. Wunsch (Ed.), Georg Thieme Verlag, Stuttgart, Ger. (1974).

Nishiuchi, Y. et al. (1993). Int. J. Pept. Protein Res. 42:533-538.

Nguyen, T.-D. et al. (2000). J. Neurosci. Res. 60:175-183.

Olivera, B. M. et al. (1985). Science 230:1338-1343.

Olivera, B. M. et al. (1990). Science 249:257-263.

Olivera, B. M. (1997). Mol. Biol. Cell 8:2101-2109.

Olivera, B. M. et al. (1999). Ann. N. Y Acad. Sci. 870:223-237.

Ornstein, et al. (1993). Biorganic Medicinal Chemistry Letters 3:43-48.

Piscano, A. et al. (1993). Glycobiology 3:429-435.

Remington's Pharmaceutical Sciences, 18th Ed., Mack Publishing Co., Easton, Pa. (1990).

Rivier, J. R. et al. (1978). Biopolymers 17:1927-38.

Rivier, J. R. et al. (1987). Biochem. 26:8508-8512.

Sambrook, J. et al. (1989). Molecular Cloning: A Laboratory Manual, 2nd Ed., Cold Spring Harbor Laboratory, Cold Spring Harbor, N.Y.

Schroder & Lubke (1965). The Peptides 1:72-75, Academic Press, NY.

Shon, K-J. et al. (1998). J. Biol. Chem. 273:33-38.

Stewart and Young, Solid-Phase Peptide Synthesis, Freeman & Co., San Francisco, Calif. (1969).

Stühmer, W. (1992) Meth. Enzymol. 207, 319-339

Terlau, H. et al. (1996). Nature 381:148-151.

Terlau, H. et al. (1999). J. Gen. Physiol. 114:125-140.

Van de Steen et al. (1998). Crit. Rev. Biochem. Mol. Biol. 33:151-208.

Woodward, S. R. et al. (1990). EMBO J. 1:1015-1020.

Yoshikami, D. et al. (1989). Ann. N.Y. Acad. Sci. 560:230-248.

Zhou L. M., et al. (1996). J. Neurochem. 66:620-628.

U.S. patent application Ser. No. 09/910,009

U.S. Pat. No. 3,842,067.

U.S. Pat. No. 3,862,925.

U.S. Pat. No. 3,972,859.

U.S. Pat. No. 4,105,603.

U.S. Pat. No. 4,447,356.

U.S. Pat. No. 4,569,967.

U.S. Pat. No. 5,364,769.

U.S. Pat. No. 5,514,774.

U.S. Pat. No. 5,534,615.

U.S. Pat. No. 5,550,050.

U.S. Pat. No. 5,591,821.

U.S. Pat. No. 5,633,347.

U.S. Pat. No. 5,635,347.

U.S. Pat. No. 5,844,077.

U.S. Pat. No. 5,889,147.

U.S. Pat. No. 6,077,934.

PCT Published Application WO 92/19125.

PCT Published Application WO 94/25503.

PCT Published Application WO 95/01203.

PCT Published Application WO 96/02286.

PCT Published Application WO 96/02646.

PCT Published Application WO 96/05452.

PCT Published Application WO 96/11698.

PCT Published Application WO 96/40871.

PCT Published Application WO 96/40979.

PCT Published Application WO 97/12635.

PCT Published Application WO 02/07678.

What is claimed is:

1. A method for regulating the flow of potassium through potassium channels in an individual in need thereof which comprises administering a therapeutically effective amount of a κM conopeptide, wherein said κM conopeptide is selected from the group consisting of:
(i) a peptide having the general formula Leu-X1-Ser-Cys-Cys-Ser-Leu-Asn-Leu-Arg-Leu-Cys-X1-Val-X1-Ala-Cys-Lys-Arg-Asn-X1-Cys-Cys-Thr (SEQ ID NO:1), wherein X1 is Pro or hydroxy-Pro and the C-terminus is amidated or a free carboxyl and
(ii) a derivative of the peptide of (i), wherein said derivative is the peptide in which the Arg residues may be substituted by Lys, ornithine, homoargine, nor-Lys, N-methyl-Lys, N,N-dimethyl-Lys, N,N,N-trimethyl-Lys or any synthetic basic amino acid; the Lys residues may be substituted by Arg, ornithine, homoargine, nor-Lys, N-methyl-Lys, N,N-dimethyl-Lys, N,N,N-trimethyl-Lys or any synthetic basic amino acid; the Ser residues may be substituted with Thr or any synthetic hydroxylated amino acid; the Thr residues may be substituted with Ser or any synthetic hydroxylated amino acid; the Asn, Ser, Thr or Hyp residues may be glycosylated; the aliphatic amino acids may be substituted by synthetic derivatives bearing non-natural aliphatic branched or linear side chains CₙH₂ₙ₊₂ up to and including n=8; the Leu residues may be substituted with Leu (D); and the Asn residues may be substituted with Gln.

2. The method of claim 1, wherein the κM conopeptide is the peptide having the general formula Leu-X1-Ser-Cys-Cys-Ser-Leu-Asn-Leu-Arg-Leu-Cys-X1-Val-X1-Ala-Cys-Lys-Arg-Asn-X1-Cys-Cys-Thr (SEQ ID NO: 1), wherein X1 is Pro or hydroxy-Pro and the C-terminus is amidated or a free carboxyl.

3. The method of claim 1, wherein said individual in need thereof suffers from a disorder selected from the group consisting of multiple sclerosis, other demyelinating diseases (such as acute dissenmiated encephalomyelitis, optic neuromyelitis, adrenoleukodystrophy, acute transverse myelitis, progressive multifocal leukoencephalopathy), sub-acute sclerosing panencephalomyelitis (SSPE), metachromatic leukodystrophy, Pelizaeus-Merzbacher disease, botulinum toxin poisoning, Huntington's chorea, compression and entrapment neurophathies (such as carpal tunnel syndrome, ulnar nerve palsy), cardiac arrhythmias, congestive heart failure, reactive gliosis, hyperglycemia, immunosuppression, cocaine addiction, cancer, cognitive dysfunction, disorders resulting from defects in neurotransmitter release (such as Eaton-Lambert syndrome), and reversal of the actions of curare and other neuromuscular blocking drugs.

4. The method of claim 1, wherein said disorder is a demyelinating disease.

5. The method of claim 1, wherein X1 is hydroxy-Pro.

6. The method of claim 5, wherein said individual in need thereof suffers from a disorder selected from the group consisting of multiple sclerosis, other demyelinating diseases (such as acute dissenmiated encephalomyelitis, optic neuromyelitis, adrenoleukodystrophy, acute transverse myelitis, progressive multifocal leukoencephalopathy), sub-acute sclerosing panencephalomyelitis (SSPE), metachromatic leukodystrophy, Pelizaeus-Merzbacher disease, botulinum toxin poisoning, Huntington's chorea, compression and entrapment neurophathies (such as carpal tunnel syndrome, ulnar nerve palsy), cardiac arrhythmias, congestive heart failure, reactive gliosis, hyperglycemia, immunosuppression, cocaine addiction, cancer, cognitive dysfunction, disorders resulting from defects in neurotransmitter release (such as Eaton-Lambert syndrome), and reversal of the actions of curare and other neuromuscular blocking drugs.

7. The method of claim 5, wherein said disorder is a demyelinating disease.

8. The method of claim 1, wherein the κM conopeptide is the derivative.

9. The method of claim 8, wherein said individual in need thereof suffers from a disorder selected from the group consisting of multiple sclerosis, other demyelinating diseases (such as acute dissenmiated encephalomyelitis, optic neuromyelitis, adrenoleukodystrophy, acute transverse myelitis, progressive multifocal leukoencephalopathy), sub-acute sclerosing panencephalomyelitis (SSPE), metachromatic leukodystrophy, Pelizaeus-Merzbacher disease, botulinum toxin poisoning, Huntington's chorea, compression and entrapment neurophathies (such as carpal tunnel syndrome, ulnar nerve palsy), cardiac arrhythmias, congestive heart failure, reactive gliosis, hyperglycemia, immunosuppression, cocaine addiction, cancer, cognitive dysfunction, disorders resulting from defects in neurotransmitter release (such as Eaton-Lambert syndrome), and reversal of the actions of curare and other neuromuscular blocking drugs.

10. The method of claim 8, wherein said disorder is a demyelinating disease.

11. The method of claim 2, wherein X1 is hydroxy-Pro.

12. The method of claim 11, wherein said individual in need thereof suffers from a disorder selected from the group consisting of multiple sclerosis, other demyelinating diseases (such as acute dissenmiated encephalomyelitis, optic neuromyelitis, adrenoleukodystrophy, acute transverse myelitis, progressive multifocal leukoencephalopathy), sub-acute sclerosing panencephalomyelitis (SSPE), metachromatic leukodystrophy, Pelizaeus-Merzbacher disease, botulinum toxin poisoning, Huntington's chorea, compression and entrapment neurophathies (such as carpal tunnel syndrome, ulnar nerve palsy), cardiac arrhythmias, congestive heart failure, reactive gliosis, hyperglycemia, immunosuppression, cocaine addiction, cancer, cognitive dysfunction, disorders resulting from defects in neurotransmitter release (such as Eaton-Lambert syndrome), and reversal of the actions of curare and other neuromuscular blocking drugs.

13. The method of claim 11, wherein said disorder is a demyelinating disease.
